(19) United States
(12) Patent Application Publication
(10) Pub. No.: US 2019/0337876 A1
(43) Pub. Date: Nov. 7, 2019

(54) INTEGRATED SYSTEM AND METHOD FOR PRODUCING METHANOL PRODUCT

(71) Applicant: AVOCET IP LIMITED, Berwick Upon Tweed (GB)

(72) Inventors: Glyn David SHORT, Hockessin, DE (US); James Robert JENNINGS, Yarm (GB)

(73) Assignee: Avocet IP Limited, Berwick Upon Tweed (GB)

(21) Appl. No.: 16/062,809

(22) PCT Filed: Dec. 15, 2016
(86) PCT No.: PCT/IB2016/001925

§ 371 Date: Jun. 15, 2018

(30) Foreign Application Priority Data
Dec. 17, 2015 (GB) 1522326.6

(51) Int. Cl. C07C 29/151; C07C 29/152; C01B 3/02

(52) U.S. Cl. CPC C07C 29/1518; C07C 31/04; C01B 3/02; C07C 29/152

(57) ABSTRACT

An improved system and method for producing methanol product. The system and the method of the present disclosure uses a gas feed arrangement for providing a methane rich gas feed and an apparatus for converting the methane rich gas feed into the methanol product. The apparatus includes an auto-thermal reforming arrangement for converting one or more gaseous components present in the methane rich gas feed. The auto-thermal reforming arrangement is configured to partially oxidise the methane rich gas feed to maintain the auto-thermal reforming arrangement at a working temperature when in operation. Also, the auto-thermal reforming arrangement employs electrical power generated from an energy source, preferably a renewable energy source, to provide additional heating in the auto-thermal reforming arrangement for maintaining the working temperature when in operation.

### TECHNICAL FIELD

The present disclosure relates to producing methanol and similar substances. In particular, the present disclosure relates to an integrated system and a method for producing methanol product from a methane rich gas, such as biogas and natural gas.

### BACKGROUND

Biogas refers to a gaseous fuel produced by the biological breakdown of organic matter in the absence of oxygen. It is produced by the anaerobic digestion or fermentation of biodegradable materials such as biomass, manure, sewage, municipal waste, green waste, plant material and crops. Biogas primarily comprises methane and carbon dioxide, and its production is well known in the prior art. Further, a prudent use of biogas can be seen in the generation of methanol.

Methanol, also known as wood alcohol, is a versatile compound which is used in industrial and house-hold products. Traditionally, methanol was produced as a by-product of the destructive distillation of wood. Nowadays, methanol is mainly produced using hydrocarbons and, in particular, methane as a raw material. In contemporary methanol production, a feedstock material is utilized to produce a synthesis gas. Accordingly, the synthesis gas is processed to convert it into methanol. Example of the feedstock material which is rich in methane includes, but may not be limited to, natural gas and biogas.

Most of known methods to produce methanol utilise natural gas and/or bio-mass as a feedstock material. These feedstock materials often contain sulphur and other noxious compounds that need to be removed. To remove sulphur compounds, Co/Mo hydrodesulphurisation, followed by a ZnO treatment, can be performed. Other techniques are also available, including absorption onto activated materials such as activated carbon, and a well-known Claus Process for sulphur removal and the like. The methods to produce synthesis gas employ moderate-pressure steam, in a steam reforming reaction, at temperatures approximately in a range of 750° C. to 900° C. Thereafter, the synthesis gas is converted into methanol. Moreover, surplus hydrogen from such processes which use methane as the principal feed gas is removed from the synthesis loop and can be exported as a separate product or used elsewhere within a given chemical complex. However, the known methods do not always make use of all the by-products produced in the synthesis of methanol. In addition, the steam reforming step of known methods utilizes combustion of some of the methane gas to satisfy thermodynamic demands of the steam reforming reaction and to produce associated required high temperatures, so that a forward reaction becomes thermodynamically favourable. Consequently, burning a significant proportion of the methane results in a substantial loss of the feedstock material.

In other prior arts, the methanol may be produced from renewable energy sources. Processes are employed which use biomass and water as main feedstock materials. Alternatively, auto-thermal reforming of natural gas or biogas can be used. However, these processes do not make use of all the by-products generated in the production of synthesis of gas. Moreover, the feedstock material is not efficiently utilized. Furthermore, a yield of methanol produced from these methods is relatively low.

The synthesis gas for catalytic methanol production is produced by a selection from gasification of biomass, CO₂ captured from post-combustion and an electrolysis of water for obtaining the hydrogen. The process of electrolysis requires energy, i.e. to produce hydrogen, which may be provided by renewable energy sources. However, presently, renewable energy contributes only about 5% of the commercial hydrogen production primarily via water electrolysis, while other 95% hydrogen is mainly derived from fossil fuels¹. Renewable hydrogen production is not popular yet because the cost is still high and the poor availability of large scale electrolysis units. Photovoltaic water electrolysis may become more competitive as the cost continues to decrease with the technology advancement; but, the considerable use of small bandgap semiconducting materials may cause serious life cycle environmental impacts. However, photocatalytic water-splitting using TiO₂ for hydrogen production offers a promising way for clean, low-cost and environmentally friendly production of hydrogen by solar energy.² ¹Ni M, Leung M K H, Sumathy K, Leung D Y C. Water electrolysis—a bridge between renewable resources and hydrogen. Proceedings of the International Hydrogen Energy forum, vol. 1, 25-28 May 2004, Beijing, PRC. p. 475-480²Ni, Meng, et al. “A review and recent developments in photocatalytic water-spitting using TiO2 for hydrogen production,” Renewable and Sustainable Energy Reviews 11.3 (2007): 401-425.

Generally, biogas to liquid fuel converters needs large amounts of equipment and investment capital. These converters require very large amounts of bio-gas at a site to justify construction, transportation and operation of large scale methanol production. The Lurgi process for low-pressure crude methanol production from bio-gas is one example of a very large scale operation.

Similarly, methane to liquid fuels processes such as the Fischer-Tropsch process have seen commercial use. However, these processes can be difficult to control and often suffer from catalyst deactivation. These processes are also only economical at very large volumes which require large initial capital investments. Therefore, none of the existing technologies provides scalable, inexpensive and reliable processes for forming hydrocarbon fuels, nor can they be deployed economically at tunable volume biogas sources. Thus, a new process, which may be an integrated system to digest waste and to utilize the biogas generated from the waste into methanol may be desirous.

In a WIPO publication WO 2012/151545 A2 (Michael S. Hsu; “Zero Emission Power Plant with CO₂ Waste Utilization”; assigned to Ztek Corporation), there is described a clean energy system, a renewable energy system or a zero emission energy system (ZEES) to utilize CO₂ waste. The energy system may include a fuel processor, an energy catalytic reactor, and a power generator. The fuel processor may catalytically convert the CH₄ component in the natural gas, biogas or syngas into a reformate including H₂, CO, CO₂ and H₂O species. The energy reactor may convert the reformate in gas form into a liquid fuel.

In a United States patent document US 2012/041083 A1 (Peter Grauer; “Silicon or Elementary Metals as Energy Carriers”; assigned to Silicon Fire Ag), there is described a method for providing storable and transportable energy carriers is described. In one step, a transformation of silicon-dioxide-containing or metal-oxide-containing starting material to silicon or a metal occurs in a reduction process, wherein the primary energy for this reduction process is provided from a renewable energy source. Then, a portion of the reaction products from the reduction process is applied in a process for generating methanol, wherein a synthesis gas composed of carbon monoxide and hydrogen is used.

In another United States patent document US 2010/022669 A1 (Cohn et al.; “Renewable Electricity Conversion of Liquid Fuels from Hydrocarbon Feedstocks”), a method for converting renewable energy source electricity and a hydrocarbon feedstock into a liquid fuel by providing a source of renewable electrical energy in communication with a synthesis gas generation unit and an air separation unit. Oxygen from the air separation unit and a hydrocarbon feedstock is provided to the synthesis gas generation unit, thereby causing partial oxidation reactions in the synthesis gas generation unit in a process that converts the hydrocarbon feedstock into synthesis gas. The synthesis gas is then converted into a liquid fuel.

In a chinese patent document CN 103952192 A (Liu Shuping; “Methanol Fuel Additive for Vehicles and Preparation Method of Methanol Fuel Additive”), there is described a methanol fuel additive for vehicles and a manufacturing method of the methanol fuel additive. The methanol fuel additive for vehicles is formed by mixing the following components in parts by weight: 9900-10200 parts of ethanol, 198-202 parts of glycol-butyl ether, 198-202 parts of ammonium acetate, 495-505 parts of linoleic acid, 247.5-252.5 parts of glycol, 495-505 parts of polysorbate-80, 2970-3030 parts of seaweed powder and 2970-3030 parts of ping tree powder.

In view of the aforementioned problems associated with known approaches, there is a need for a system and method that makes efficient use of feedstock material and renewable solar or wind energy source. In addition, the system and the method should make use of available alternatives for satisfying endothermic demands of the reaction. Moreover, the system and method should be able to achieve the maximum possible yield of the required products including, but may not be limited to, synthesis gas and methanol. Furthermore, the system and method should make use of renewable or agricultural by-products without producing unwanted by-products using clean, low-cost and environmentally friendly production of hydrogen from renewable energy.

### SUMMARY

The present disclosure seeks to provide an integrated system for producing a methanol product.

Moreover, the present disclosure seeks to provide an improved method for producing a methanol product using an integrated system having a gas feed arrangement and an apparatus for converting a gas feed from the gas feed arrangement into the methanol product.

According to a first aspect of this disclosure, there is provided an integrated system for producing a methanol product, the system comprising:

a gas feed arrangement for providing a methane rich gas feed; and
an apparatus for converting the methane rich gas feed into the methanol product, the apparatus comprising:
   an auto-thermal reforming arrangement for converting one or more gaseous components present in the methane rich gas feed, wherein the auto-thermal reforming arrangement is configured to partially oxidise the methane rich gas feed to maintain the auto-thermal reforming arrangement at a working temperature when in operation, characterized in that the auto-thermal reforming arrangement also employs electrical power generated from an energy source, preferably a renewable energy source, to provide additional heating in the auto-thermal reforming arrangement for maintaining the working temperature when in operation;
   a synthesis arrangement for producing methanol product from synthesis gas, hydrogen and carbon dioxide;
   an oxygen feed for supplying oxygen to the auto-thermal reforming arrangement;
   an electrolysing arrangement for electrolysing water to provide the oxygen feed, and hydrogen feed for the synthesis arrangement; and
   a continuous flow for adding an additive to the methanol product to constitute a liquid fuel.

In one embodiment of the present disclosure, the gas feed arrangement comprises an organic waste material digestion arrangement for providing biogas as the methane rich gas feed. Alternatively, the gas feed arrangement comprises a natural gas source for providing natural gas, such as by ‘anaerobic digestion’ (AD) which produces a methane-rich gas produced by fermentation of biomass.

In an embodiment of the present disclosure, the apparatus includes a gas purification and separating arrangement for purifying the methane rich gas feed to remove sulphur components therein and for separating the purified gas feed into at least carbon dioxide and methane component, wherein the methane component is provided to the auto-thermal reforming arrangement for producing a synthesis gas, and the apparatus is operable to react the synthesis gas with the carbon dioxide component in a synthesis arrangement to produce the methanol product.

In an embodiment of the present disclosure, the apparatus includes an oxygen feed to an auto-thermal reforming arrangement.

In one embodiment of the present disclosure, the renewable energy source comprises at least one of solar energy, wind energy, geothermal energy, hydroelectricity and tidal energy.

In an embodiment of the present disclosure, the apparatus further comprises one of an agitation tank, a gas bubbling tank and a continuous flow for adding an additive to the methanol product to constitute a liquid fuel. For example, the additive comprises at least one of polyethylene glycol dinitrate, ammonium nitrate, urea, Avocet™ or any combination thereof.

According to a second aspect, there is provided a method for producing methanol product using integrated system having a gas feed arrangement and an apparatus for converting a gas feed from the gas feed arrangement into the methanol product, characterized in that the method comprising:
collecting a methane rich gas feed from the gas feed arrangement;
using an auto-thermal reforming arrangement of the apparatus for converting one or more gaseous components present in the methane rich gas feed;
arranging for the apparatus to partially oxidise the methane rich gas feed to maintain the auto-thermal reforming arrangement at a working temperature when in operation;
arranging for the apparatus to employ electrical power generated from an energy source, preferably a renewable energy source, to provide additional heating in the auto-thermal reforming arrangement for maintaining the working temperature when in operation
using a synthesis arrangement to produce methanol product from synthesis gas, hydrogen and carbon dioxide;
using an oxygen feed of the apparatus for supplying oxygen to the auto-thermal reforming arrangement;
using an electrolysing arrangement of the apparatus for electrolysing water to provide the oxygen feed and a hydrogen feed for the synthesis arrangement; and
using a continuous flow for adding an additive to the methanol product to constitute a liquid fuel.

In an embodiment of the present disclosure, the method further comprises using a gas purification and separating arrangement of the apparatus for purifying the methane rich gas feed to remove sulphur components therein, and for separating the purified gas feed into at least carbon dioxide and methane components; and providing the methane component to the auto-thermal reforming arrangement for producing a synthesis gas, and reacting the synthesis gas with the carbon dioxide component in a synthesis arrangement to produce the methanol product.

In one embodiment of the present disclosure, the method further comprises adding an additive to the methanol product to constitute a liquid fuel. For example, the additive comprises at least one of polyethylene glycol dinitrate, ammonium nitrate, urea, Avocet or any combination thereof.

In an embodiment of the present disclosure, the methane rich gas feed is one of a biogas derived from anaerobic digestion of slurry, partial ovidation of biomass such as wood chips, or any similar source.

In one embodiment of the present disclosure, the renewable energy source comprises at least one of solar energy, wind energy, geothermal energy, hydroelectricity and tidal energy.

In a typical auto-thermal reforming utilizing a reactor, highest temperatures in the reactor are close to an inlet of the reactor and lowest temperatures are encountered at an outlet of the reactor. This is disadvantageous because an equilibrium constant for the steam reforming reaction becomes more favourable at the higher temperatures than at the lower temperatures. Thus, some of the methane feedstock is not converted to synthesis gas in a typical known reactor. In an embodiment of the present disclosure, the apparatus is operable to employ supplementary electrical power generated from a renewable energy source to provide additional heating in the auto-thermal reforming arrangement to maintain the exit gas at its optimum working temperature during production of methanol. For example, the partial oxidation of the methane rich gas feed may maintain a required high temperature at an inlet of the auto-thermal reforming arrangement, and the heating provided by the electrical power may maintain a required high temperature at an outlet of the auto-thermal reforming arrangement.

The present disclosure is of advantage in that the capital costs used in the operation stated in the disclosure are very low in comparison to other processes using conventional steam reforming. In the present disclosure, biogas is beneficially used as a feedstock material. The biogas can easily be generated from biomass. In addition, other inputs, such as hydrogen and oxygen, for improving the conversion of biogas into methanol are generated using renewable energy sources. Moreover, an auto-thermal reforming is done to convert methane gas to synthesis gas. The auto-thermal reforming uses oxygen, steam and carbon dioxide as inputs to react with methane over a catalyst. Furthermore, the addition of supplementary heat to an exit part of the auto-thermal reforming arrangement results in a reduction of the amount of methane that remains unconverted to synthesis gas.

In particular, the advantage of the integrated system for generation of biogas and methanol is the optimal use of the feedstock material in the farm situation. Methane rich gas from wood chips, hydroponics, and farm waste is channelled into the apparatus to generate methanol. Costs due to location and logistics are minimized. Further, in the apparatus to generate methanol, the temperature of the auto-thermal reforming arrangement is stabilised for optimal methanol production by minimising the temperature difference between the inlet and outlet. In a conventional system the methanol production gradually decreases due to a decrease in temperature within the length of the auto-thermal reforming arrangement from the inlet to the outlet. Usually electricity is used to prevent such a fall in temperature, but prohibitive costs make it a non-viable solution. In this invention the use of renewable energy provideenergy, supplementary to that released during the autothermal reforming stage to maintain temperature uniformity and a steady generation of methanol thus making it a far more efficient system.

It will be appreciated that features of the present disclosure are susceptible to being combined in various combinations without departing from the scope of the invention as defined by the appended claims.

### DESCRIPTION OF THE DIAGRAMS

Embodiments of the present disclosure will now be described, by way of example only, with reference to the following diagrams wherein:

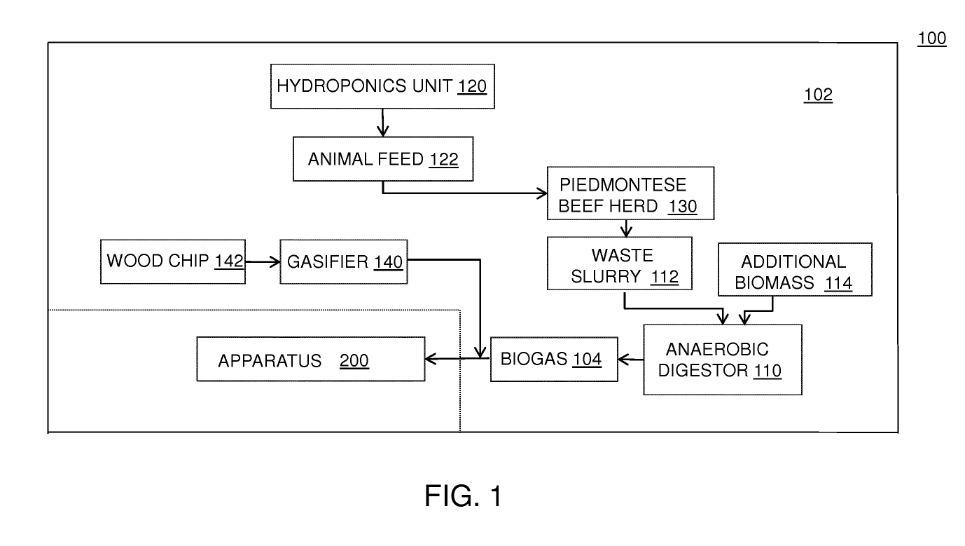

FIG. 1 is an illustration of various functional components of an integrated system for producing methanol product, in accordance with various embodiments of the present disclosure;

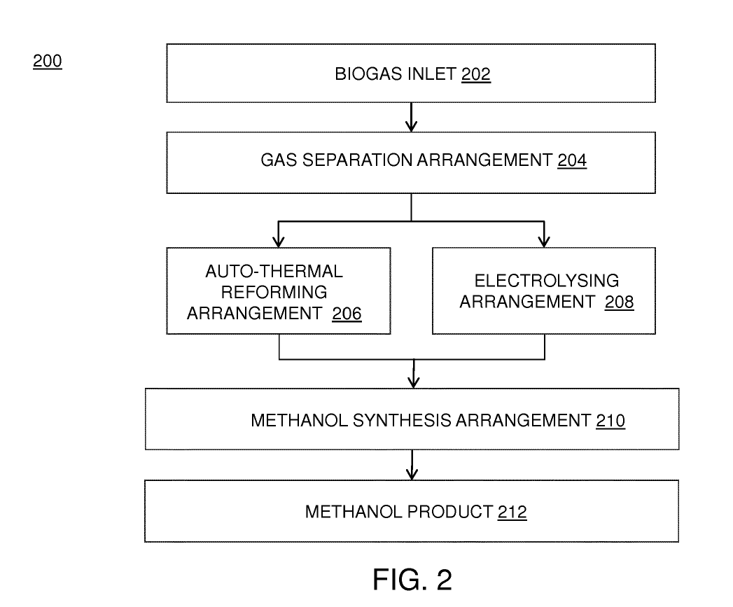

FIG. 2 is an illustration of various functional components of an apparatus of the integrated system of FIG. 1, in accordance with various embodiments of the present disclosure;

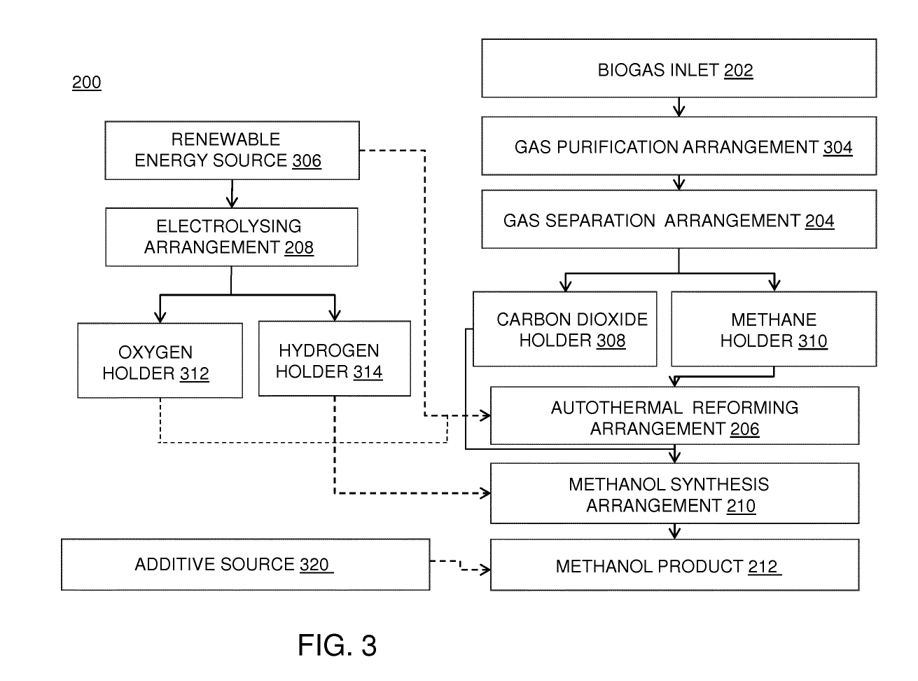

FIG. 3 is an illustration of a detailed view of the apparatus for converting a methane rich gas feed into a methanol product, in accordance with various embodiments of the present disclosure;

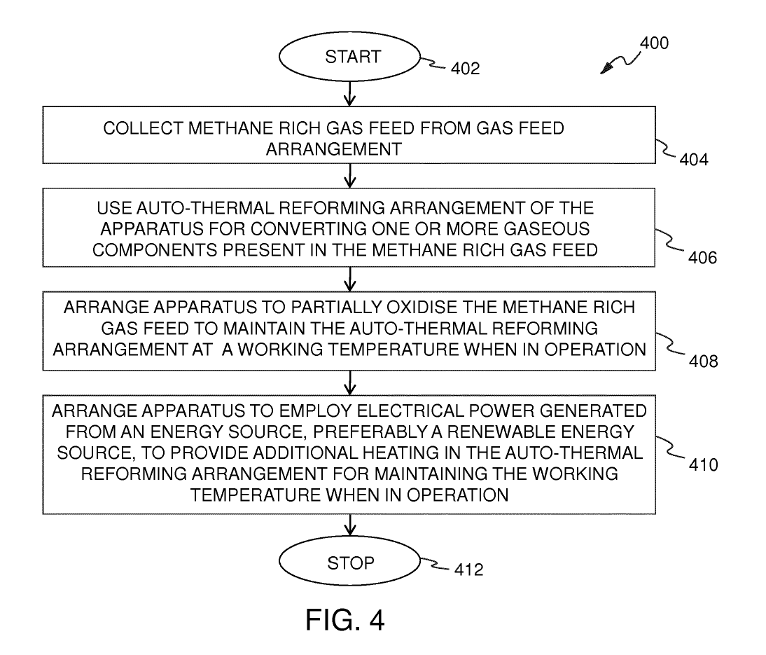

FIG. 4 is an illustration of a method for using the integrated system for producing methanol product, in accordance with an embodiment of the present disclosure;

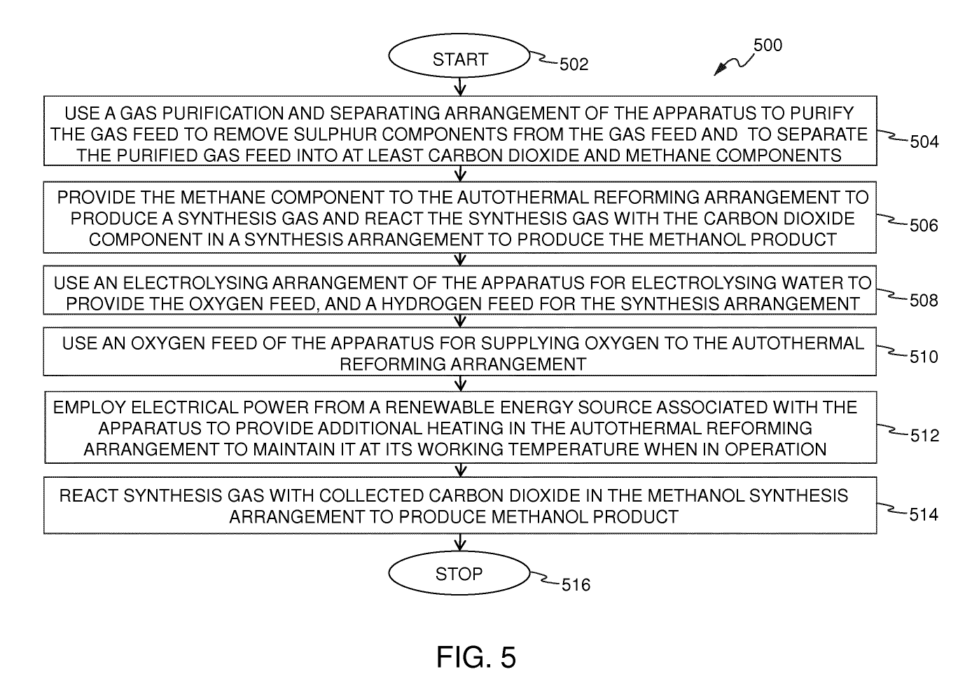

FIG. 5 is an illustration of a detailed flowchart for using an apparatus of the integrated system for converting the methane rich gas feed into the methanol product, in accordance with another embodiment of the present disclosure; and

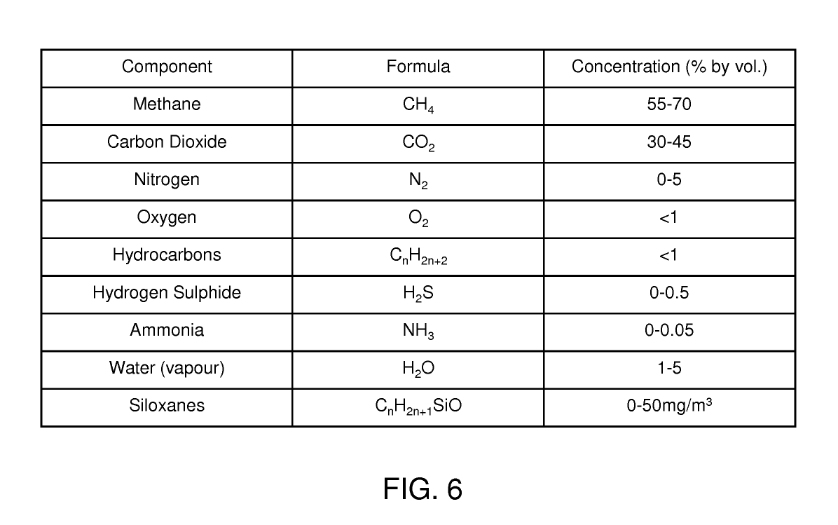

FIG. 6 is an illustration of a typical composition of a methane rich gas, such as biogas.

In the accompanying diagrams, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DESCRIPTION OF EXAMPLE EMBODIMENTS

FIG. 1 is an illustration of various functional components of an integrated system 100 for producing methanol product, in accordance with various embodiments of the present disclosure. The integrated system 100 includes a gas feed arrangement 102 for providing a methane rich gas feed, such as biogas 104. The integrated system 100 also includes an apparatus 200 for converting the gas feed from the gas feed arrangement 102 into the methanol product. The gas feed arrangement 102 produces the biogas 104 by anaerobic digestion in a digestor 110 of waste slurry 112 and additional biomass 114. For example, the waste slurry 112 may derive material from a hydroponics unit 120 providing animal feed 122. In the present embodiment, the hydroponics unit 120 is the production system used to grow cattle feed (i.e. animal feed 122, for example on which a piedmontese beef herd 130 feeds to produce biomass. In one embodiment, the gas feed arrangement 102 also includes a gasifier 140 for cleaning methane rich gas generated from wood chips 142, and is further connected to the main biogas gas 104 feed to the apparatus 200.

The biogas 104 generated from the anaerobic digestor 110 (as well as the methane rich gas provided by the gasifier 140) is fed into the apparatus 202 which has an auto-thermal reforming arrangement for converting one or more gaseous components present in the gas feed into methanol product. In the present embodiment, the gas feed arrangement 102 is an organic waste material digestion arrangement for providing biogas 104 as the methane rich gas feed. For example, the organic waste material digestion arrangement may include a digester unit (such as digestor 110) and a technical unit (not shown) for the control of digestion parameters and composition adjustment for the production of biogas.

In another embodiment, the apparatus 200 is arranged to operate another gas feed arrangement, such as a natural gas source that providing natural gas as the methane rich gas feed. For example, the natural gas can be AD gas.

FIG. 2 is an illustration of an overview of different functional arrangements of an apparatus 200 for producing methanol from biogas included in a gas feed, in accordance with various embodiments of the present disclosure. The apparatus 200 is optionally an industrial plant set-up having different arrangements for performing different processes. The apparatus 200 includes a biogas inlet 202 (to be operatively coupled with the gas feed arrangement 102, for example the biogas 104 holder of FIG. 1). The apparatus 200 also includes a gas separation arrangement 204, an auto-thermal reforming arrangement 206, an electrolysing arrangement 208 and a methanol synthesis arrangement 210. The biogas inlet 202 receives biogas (i.e. methane rich gas feed) from the gas feed arrangement 102, and the gas separation arrangement 204 separates contents of methane rich gas feed and extracts carbon-dioxide and methane. A typical composition of biogas (shown in a table 600 of FIG. 6) includes a gaseous mixture of methane, carbon dioxide (CO₂), nitrogen, oxygen, hydrocarbons, hydrogen sulphide, ammonia, water vapour, siloxanes, and the like. Accordingly, the auto-thermal reforming arrangement 206 utilizes the methane including similar light hydrocarbons from the biogas to produce synthesis gas. In addition, the electrolysing arrangement 208 electrolyses water to produce hydrogen and oxygen used for producing methanol. Furthermore, the methanol synthesis arrangement 210 utilizes hydrogen, carbon dioxide and synthesis gas to produce methanol product 212, described in detail with reference to FIG. 3.

It will be appreciated that the apparatus 200 shown in FIG. 1 uses biogas as a feedstock material; however, it will also be appreciated that natural gas (such as AD gas) or methane from any appropriate source may be used as a feedstock material in the absence of biogas. In addition, the auto-thermal reforming arrangement 206 may be fully or partially replaced with the steam reforming arrangement. Moreover, the electrolysing arrangement 208 may be replaced by externally-provided oxygen and hydrogen cylinders.

FIG. 3 is an illustration of a detailed view of different functional arrangements of the apparatus 200 for converting a gas feed into the methanol product 212, in accordance with various embodiments of the present disclosure. The apparatus 200 includes a gas purification arrangement 304, a carbon-dioxide holder 308, a methane holder 310, an oxygen holder 312 and a hydrogen holder 314 apart from the biogas inlet 202, the gas separation arrangement 204, the auto-thermal reforming arrangement 206, the methanol synthesis arrangement 210 and the electrolysing arrangement 208, also shown in FIG. 2.

As aforementioned, the apparatus 200 may be an industrial plant set-up having different arrangements for performing different processes. For example, the biogas inlet 202 may be provided by the gas feed arrangement 102, shown in FIG. 1, responsible for generating biogas from biomass. The gas purification arrangement 304 purifies the generated biogas to remove sulphur compounds therefrom. In an embodiment of the present disclosure, the sulphur components may be removed using conventional ZnO technology or some other known technology or processes. After the purification, the gas separation arrangement 204 separates methane and carbon-dioxide from the gas feed. The carbon-dioxide holder 308 collects the separated carbon-dioxide and the methane holder 310 collects separated methane. Accordingly, the auto-thermal reforming arrangement 206 converts methane to synthesis gas. In an embodiment, the auto-thermal reforming arrangement 206 utilizes energy from different energy sources which include, but may not be limited to, a renewable energy source 306. The renewable energy source 306 includes at least one of solar energy, wind energy, geothermal energy and tidal energy.

The electrolysing arrangement 208 simultaneously electrolyses water to produce oxygen and hydrogen. The oxygen holder 312 and the hydrogen holder 314 collect oxygen and hydrogen respectively. Part or all of the collected oxygen is supplied to the inlet of the auto-thermal reforming arrangement 206 to react with methane to generate sufficient heat, so that, in the presence of added steam, the methane is converted over the reforming catalyst to produce synthesis gas. The synthesis gas, rich in hydrogen, reacts with collected carbon dioxide in the methanol synthesis arrangement 210 to produce the methanol product 212. In an embodiment, hydrogen may be fed to the methanol synthesis arrangement 210 to react with any additional carbon dioxide obtained from the separation stage and hence increase an overall yield of methanol product 212 from a given quantity of biogas.

In an example, methanol synthesis gas is characterised by the stoichiometric ratio (H₂—CO₂)/(CO+CO₂), often referred to as the module M. A module of 2 defines a stoichiometric synthesis gas for formation of methanol. Further, the process is based on known chemistry but the methane is separated and purified (and steam reformed (known) using autothermal reformers to produce synthesis gas. The reactions of the synthesis process are:

CH₄+H₂O=>CH₃OH+H₂ (1)

CH₄+H₂O=>CO+3H₂ (2)

CH₄+2H₂O=>+CO₂+4H₂ (3)

Reaction (2) of catalytic methane steam reforming is strongly endothermic so that steam reforming for production of methanol requires an external heat supply. In this invention the reaction can be run much closer to stoichiometric ratios, allowing more carbon content from the methane rich gas (AD gas or biogas) to produce Methanol, thus obtaining more methanol for the same amount of methane input. The composition of the synthesis gas is too rich in Hydrogen for stoichiometric methanol synthesis so by adding in some of the separated CO₂ the stoichiometric ratios of methanol are balanced.

It will be appreciated that both the auto-thermal reforming reaction and the steam reforming reaction require the addition of steam as a key reactive component in the reaction; however, the auto-thermal reforming reaction retains the methane as a carbon source and does not burn it externally. Hence, the oxidation products from the methane are not lost to atmosphere.

It will be further appreciated that the apparatus 200 shown in FIG. 2 removes sulphur compounds from the gas feed by the gas purification arrangement 304; however, it will be appreciated that the removal of sulphur can be done by ZnO technology or some other conventional techniques. In addition, the auto-thermal reforming arrangement 206 may be fully or partially replaced by conventional steam reforming.

Moreover, the heat required for various processes may be generated by various sources including burning of methane, electrical induction and the like. Furthermore, carbon dioxide may be utilized to make use of the excess hydrogen generated during the production of synthesis gas 212. Furthermore, oxygen and carbon dioxide used in the processes may be supplied externally.

In one embodiment, the apparatus 200 further includes an additive source 320. In an example, the additive source 320 includes one of an agitation tank, a gas bubbling tank and a continuous flow for adding an additive to the methanol product to constitute a liquid fuel. For example, the additive includes at least one of polyethylene glycol dinitrate, ammonium nitrate, urea, Avocet (Avocet is a registred trade mark in the United Kingdom) or any combination thereof. The additive source 320 provides additive, which may be mixed with the methanol product 212 to constitute the liquid fuel, for example a diesel replacement.

Although the composition of Avocet is proprietary, and may have varied over time, the composition of the original Avocet additive includes following components as provided below:

PEG (PolyEthyleneGlycol) dinitrate—80%

Methanol—18%

Lubricity additive—1.5%

Antioxidant—0.1%

FIG. 4 is an illustration of a method 400 for using an integrated system (such as the integrated system 100 of FIG. 1) for producing methanol product, in accordance with an embodiment of the present disclosure. As mentioned above, the integrated system includes a gas feed arrangement (such as gas feed arrangement 102) and an apparatus (such as the apparatus 200) for converting a gas feed from the gas feed arrangement into the methanol product. At a step 402, the flowchart 400 initiates. At a step 404, as aforementioned, a methane rich gas feed is collected from the gas feed arrangement. At a step 406, an auto-thermal reforming arrangement of the apparatus is used for converting one or more gaseous components present in the methane rich gas feed. At a step 408, the apparatus is arranged to partially oxidise the methane rich gas feed to maintain the auto-thermal reforming arrangement at a working temperature when in operation. At a step 410, the apparatus is arranged to employ electrical power generated from an energy source, preferably a renewable energy source, to provide additional heating in the auto-thermal reforming arrangement for maintaining the working temperature when in operation. The flowchart 400 terminates at a step 412.

FIG. 5 is an illustration of a detailed flowchart 500 for using an apparatus (such as the apparatus 200 of FIG. 3) of the integrated system for converting the methane rich gas feed into the methanol product, in accordance with another embodiment of the present disclosure. The flowchart 500 initiates at a step 502. At a step 504, a gas purification arrangement 304 purifies the gas feed (i.e. methane rich gas feed provide by the gas feed arrangement 102) to remove sulphur components from the gas feed. In addition, the gas separating arrangement 204 of the apparatus 200 separates the purified gas feed into carbon dioxide and methane components. Following the step 504, at a step 506, the methane component is fed to the auto-thermal reforming arrangement 206 to produce a synthesis gas. Moreover, the hydrogen-rich synthesis gas reacts with the carbon dioxide component in the methane synthesis arrangement 210 to produce the methanol product 212. Following the step 506, at a step 508, the electrolysing arrangement 208 electrolyses water to provide the oxygen feed and the hydrogen feed for the methane synthesis arrangement 210. Following the step 508, at a step 510, the oxygen feed supplies oxygen to the auto-thermal reforming arrangement 206. Following the step 510, at a step 512, electrical power from a renewable energy source provides additional heating in the auto-thermal reforming arrangement 210 to maintain the required working exit temperature during the operation to minimise the methane loss due to lower conversion to synthesis gas. The renewable energy sources used to provide heating and operating the electrolysing arrangement 208 include, but may not be limited to, solar and wind generators. Simultaneously, the electrolysing arrangement 208 electrolyses water to produce oxygen and hydrogen. The oxygen holder 312 and the hydrogen holder 314 collect oxygen and hydrogen respectively. The collected oxygen is supplied to the auto-thermal reforming arrangement 206 to react with methane in the presence of catalyst to form synthesis gas. At a step 514, the synthesis gas reacts with collected carbon dioxide in the methanol synthesis arrangement 210 to produce methanol product 212. At a step 516, the flowchart 500 terminates.

In one embodiment, the flowchart 500 may include addition of an additive to the methanol product to constitute a liquid fuel, for example using the additive source 320. For example, the additives may include at least one of polyethylene glycol dinitrate, ammonium nitrate, urea, Avocet™ or any combination thereof. Specifically, the additive may be mixed with the methanol product to constitute the liquid fuel, for example, a diesel replacement.

The different functional arrangements of the integrated system 100 explained in conjunction of description of FIGS. 1-3 for converting gas feed into the methanol product 212 provide many benefits over known apparatus and methods for methanol production. The integrated system 100 employs integration into a single process concept, whereby simply using biogas, water and a source of electricity, a liquid fuel is produced that makes use of all of by-products of an agricultural facility, for example a farm, without a disadvantage of simultaneously forming unwanted by-products.

In addition, steam reforming, is a highly endothermic reaction. However, in the case of auto-thermal reforming, for example by using auto-thermal reforming arrangement 206 of the apparatus 200, all the heat is generated at an inlet of a catalytic converter and gas flowing through the catalytic converter gradually cools down as it proceeds through a catalyst bed of the catalytic converter. Thus, an exit temperature of the catalytic converter is significantly lower than the inlet temperature. The conversion of methane to synthesis gas is strongly favoured by high temperatures, thus the equilibrium conversion of methane to synthesis gas gradually falls as the temperature falls. As a result, the conversion to synthesis gas is lower in an auto-thermal reforming arrangement 206 than would be the case in a conventional steam reformer. Moreover, the capital cost benefit associated with the auto-thermal reforming arrangement 206 is eroded by the lower conversion to product. However, owing to the surplus electrical energy, the temperature at the outlet of the auto-thermal reforming arrangement 206 is maintained by a form of electrical heating to achieve efficient use of some of the energy that might otherwise not be used. In addition, the exit temperature of the gaseous products is increased to achieve a maximum possible yield of synthesis gas, and therefore methanol, from the resources at disposal for use by the apparatus 200.

In addition, the apparatus 200 of the present disclosure focuses at a reforming stage employed in the methanol production. Conventional steam reforming uses externally fired reaction tubes. In this instance, some of the methane feed is simply burnt to heat the reaction tubes to sufficiently high reaction temperatures, and to provide sufficient energy to satisfy the thermodynamic demands of the endothermic steam reforming reaction occurring within the reaction tubes. Virtually all contemporary known methanol production plants employ high-temperature reaction tubes for implementing steam reforming. In an agrarian farm location, a huge amount of gas is often not available. The burning of methane potentially results in an unacceptable loss of feedstock material. Hence, at least, the reaction tubes can be optionally heated electrically using infrared or induction heating. However, pursuant to the present disclosure, a more advantageous implementation utilizes “auto-thermal reforming”.

In addition, auto-thermal reforming used in the apparatus 200 is advantageous on account of being susceptible to being implemented at a lower capital expenditure in comparison to known conventional reforming. In auto-thermal reforming, oxygen and steam are added to a methane feed to generate a gas mixture. The gas mixture is then passed over a catalyst heated to a suitable temperature. Some of the methane is burned internally in a spatial proximity of the catalyst to produce carbon monoxide (CO) and the heat thereby generated is sufficient to sustain the steam reforming reaction at the catalyst. Sufficient oxygen is provided to allow for an exit temperature in an order of 775° C., for example in a range of 750° C. to 800° C. Beneficially, such a reforming reaction is implemented in an equilibrium state, and a product generated from such an auto-thermal reforming process is identical to the product obtained by employing a conventional known steam reforming process. The use of oxygen, rather that air, is essential and a suitable supply is provided when implementing apparatus pursuant to the present disclosure.

Moreover, the production of methanol from synthesis gas derived from methane via steam reforming always results in an excess of hydrogen in a reactor loop of associated apparatus. In a conventional chemical works, such an excess of hydrogen is normally exported to another process that requires hydrogen, such as an ammonia plant and so forth. In a rural agrarian situation, for example a farm location, such other processes such as ammonia production would not be implemented, thereby raising an issue of employing the excess hydrogen in an economically beneficial manner. However, in many rural situations, a source of carbon dioxide is often available, for example arising from biogas generation. Some of the carbon dioxide is advantageously blended back into the methanol synthesis loop employed, to make best use of the excess hydrogen. In many situations, excess of hydrogen is likely to be insufficient to be able to use all of the carbon dioxide. Thus, a significant quantity of additional hydrogen must beneficially be generated separately, if optimal use of all of the available carbon dioxide is to be achieved. Such additional hydrogen is optionally beneficially obtained by electrolysis; such electrolysis has an additional advantage in providing oxygen for use in the auto-thermal reformer.

Modifications to embodiments of the present disclosure described in the foregoing are possible without departing from the scope of the present disclosure as defined by the accompanying claims. Expressions such as “including”, “comprising”, “incorporating”, “consisting of”, “have”, “is” used to describe and claim the present disclosure are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

1.-18. (canceled)

19. An integrated system for producing a methanol product, the system comprising:
a gas feed arrangement for providing a methane rich gas feed; and
an apparatus for converting the methane rich gas feed into the methanol product, the apparatus comprising:
an auto-thermal reforming arrangement for converting one or more gaseous components present in the methane rich gas feed, wherein the auto-thermal reforming arrangement configured to partially oxidise the methane rich gas feed to maintain the auto-thermal reforming arrangement at a working temperature when in operation, characterized in that the auto-thermal reforming arrangement employs electrical power generated from an energy source, preferably a renewable energy source, to provide additional heating in the auto-thermal reforming arrangement for maintaining the working temperature when in operation;
a synthesis arrangement for producing methanol product from synthesis gas, hydrogen and carbon dioxide;
an oxygen feed for supplying oxygen to the auto-thermal reforming arrangement;
an electrolysing arrangement for electrolysing water to provide the oxygen feed, and hydrogen feed for the synthesis arrangement; and
a continuous flow for adding an additive to the methanol product to constitute a liquid fuel.

20. An integrated system as claimed in claim 19, wherein the gas feed arrangement comprises an organic waste material digestion arrangement for providing biogas as the methane rich gas feed.

21. An integrated system as claimed in claim 19, wherein the gas feed arrangement comprises a natural gas source for providing natural gas as the methane rich gas feed.

22. An integrated system as claimed in claim 19, wherein the apparatus comprises a gas purification and separating arrangement for purifying the methane rich gas feed to remove sulphur components therefrom, and for separating the purified gas feed into at least carbon dioxide and methane component, wherein the methane component is provided to the auto-thermal reforming arrangement for producing a synthesis gas, and the apparatus is operable to react the synthesis gas with the carbon dioxide component in the synthesis arrangement to produce the methanol product.

23. An integrated system as claimed in claim 19, wherein the renewable energy source comprises at least one of solar energy, wind energy, geothermal energy, hydroelectricity and tidal energy.

24. An integrated system as claimed in claim 19, wherein the apparatus further comprises one of an agitation tank and a gas bubbling tank.

25. An integrated system as claimed in claim 19, wherein-the additive comprises at least one of polyethylene glycol dinitrate, ammonium nitrate, urea, or any combination thereof.

26. A method for producing a methanol product using an integrated system having a gas feed arrangement and an apparatus for converting a gas feed from the gas feed arrangement into the methanol product, the method comprising:
collecting a methane rich gas feed from the gas feed arrangement;
using an auto-thermal reforming arrangement of the apparatus for converting one or more gaseous components present in the methane rich gas feed;
arranging for the apparatus to partially oxidise the methane rich gas feed to maintain the auto-thermal reforming arrangement at a working temperature when in operation;
arranging for the apparatus to employ electrical power generated from an energy source, preferably a renewable energy source, to provide additional heating in the auto-thermal reforming arrangement for maintaining the working temperature when in operation;
using a synthesis arrangement to produce methanol product from synthesis gas, hydrogen and carbon dioxide;
using an oxygen feed of the apparatus for supplying oxygen to the auto-thermal reforming arrangement;
using an electrolysing arrangement of the apparatus for electrolysing water to provide the oxygen feed and a hydrogen feed for the synthesis arrangement; and
using a continuous flow for adding an additive to the methanol product to constitute a liquid fuel.

27. A method as claimed in claim 26, wherein the method further comprises:
using a gas purification and separating arrangement of the apparatus for purifying the methane rich gas feed to remove sulphur components therefrom, and for separating the purified gas feed into at least carbon dioxide and methane components; and
providing the methane component to the auto-thermal reforming arrangement for producing a synthesis gas, and reacting the synthesis gas with the carbon dioxide component in the synthesis arrangement to produce the methanol product.

28. A method as claimed in claim 26 wherein the additive comprises at least one of polyethylene glycol dinitrate, ammonium nitrate, urea, or any combination thereof.

29. A method as claimed in claim 26 wherein the methane rich gas feed is one of a biogas and a natural gas.

30. A method as claimed in claim 26 wherein the renewable energy source comprises at least one of solar energy, wind energy, geothermal energy and tidal energy.
